(19) United States
(12) Patent Application Publication
(10) Pub. No.: US 2004/0126339 A1
(43) Pub. Date: Jul. 1, 2004

(54) Sunscreen composition and methods for manufacturing and using a sunscreen composition

(76) Inventor: James A. Roszell, Henderson, NV (US)

Correspondence Address: MERCHANT & GOULD PC, P.O. BOX 2903, MINNEAPOLIS, MN, 55402-0903, US

(21) Appl. No.: 10/335,192

(22) Filed: Dec. 31, 2002

(51) Int. Cl. A61K 7/42; A61K 7/44

(52) U.S. Cl. 424/59; 424/60

(57) ABSTRACT

A sunscreen composition is provided. The sunscreen composition includes a mixture of a skin bonding polymer composition comprising hydrophobic polymer/hydrophilic polymer adduct, at least one sunscreen active ingredient in an amount effective to provide the sunscreen with an SPF value of at least 4, and water in an amount effective to provide the composition with a texture suitable for application to skin. The hydrophobic polymer/hydrophilic polymer adduct can be prepared by melt processing a hydrophobic polymer composition that includes repeating pyrrolidone/alkylene groups wherein the alkylene groups contain at least 10 carbon atoms, and a hydrophilic polymer composition including repeating carboxylic groups and/or repeating hydroxyl groups. Methods for manufacturing and using a sunscreen are provided.

### FIELD OF THE INVENTION

The invention relates to a sunscreen composition and to methods for manufacturing and using a sunscreen composition.

### BACKGROUND OF THE INVENTION

Sunscreen compositions are often applied to skin to protect the skin from sunburn caused by the sun's ultraviolet rays. Sunscreens can be highly effective in protecting against sunburn. Sunscreens have a tendency to wear off or dissolve away when the wearer enters water and/or as a result of perspiration. Sunscreens have been developed to resist water and perspiration.

Sunscreens are often classified by the SPF (sun protection factor) rating system that was developed to provide consumer guidance in selecting sunscreens. In general, the SPF number approximately corresponds to the multiple of time during which the sunscreen will prevent obvious reddening of the skin, over the exposure time that causes unprotected skin to exhibit reddening. Thus, a person should be able to remain in the sun without visible effect for 15 times the usual unprotected duration, if an SPF 15 sunscreen formulation has been properly applied. The exposure needed to produce a visible effect varies from individual to individual, due to differences in their skin cells.

Numerous sunscreens are described in the patent literature. For example, see U.S. Pat. No. 6,290,936 to Ross et al., U.S. Pat. No. 5,047,232 to Kaplan, U.S. Pat. No. 5,747,011 to Ross et al., U.S. Pat. No. 5,914,102 to Fowler et al., U.S. Pat. No. 6,042,813 to Fowler, U.S. Pat. No. 6,048,517 to Kaplan, and U.S. Pat. No. 6,099,825 to McShane et al.

Compositions have been developed that bond to skin and hold active ingredients in place. For example, see U.S. Patent Publication No. US-2002-0051797-A1 that is assigned to Skinvisible Pharmaceuticals, Inc., the assignee of this patent application.

### SUMMARY OF THE INVENTION

A sunscreen composition is provided according to the invention. The sunscreen composition includes a skin bonding polymer composition comprising a hydrophobic polymer/hydrophilic polymer adduct, at least one sunscreen ingredient in an amount effective to provide the sunscreen composition with an SPF value of at least 14, and water in an amount effective to provide the composition with a consistency for application to skin.

The hydrophobic polymer/hydrophilic polymer adduct can be provided by melt mixing a hydrophobic polymer composition and a hydrophilic polymer composition. The hydrophobic polymer composition can include repeating pyrrolidone/alkylene groups wherein the alkylene groups contain at least 10 carbon atoms. The hydrophilic polymer composition can contain repeating carboxylic groups and/or repeating hydroxyl groups.

The skin bonding polymer composition includes the hydrophobic polymer/hydrophilic polymer adduct, and can include additional components including water and stabilizing and/or preserving components. The skin bonding polymer composition can include at least about 50 wt. % of the hydrophobic polymer/hydrophilic polymer adduct, and can include up to 100% of the hydrophobic polymer/hydrophilic polymer adduct.

The sunscreen composition can include additional components including thickeners, preservatives, humectants, skin healing agents, pH adjusting agents, emollients, and anti-oxidants. The sunscreen composition can be provided so that it does not include organic solvents, surfactants, and silicones, or includes less of these components relative to many commercially available sunscreens. In general, the sunscreen can include less than 5 wt. % organic solvent such as an alcohol including ethyl alcohol, less than about 0.5 wt. % surfactant, and less than about 1 wt. % silicone.

A method for manufacturing a sunscreen composition is provided according to the invention. The method includes a step of mixing the skin bonding polymer composition, the at least one sunscreen active ingredient, and the water component to provide the sunscreen composition. The step of mixing can include mixing with additional components including at least one of a thickener, preservative, humectant, skin healing agent, pH adjusting agent, emollient, and anti-oxidant.

A method for using a sunscreen composition is provided according to the invention. The method includes a step of applying a sunscreen composition to skin. The step of applying can include spray application of the sunscreen composition to skin, and can include a step of hand application of the sunscreen composition to skin. In general, it is expected that the sunscreen can be provided with a viscosity that allows a user to spray the sunscreen onto skin. In addition, the sunscreen can be provided as a lotion that allows a user to squirt the sunscreen onto skin and massage it into the skin.

### DETAILED DESCRIPTION OF THE INVENTION

A sunscreen composition is provided by the invention. A sunscreen composition is a composition that, when applied to skin, protects the skin from ultraviolet radiation from the sun. The sunscreen composition can be referred to more simply as the sunscreen or as the composition.

The sunscreen includes a skin bonding polymer composition, at least one sunscreen active ingredient, and water. The sunscreen can include additional components such as those components found in skin lotions and/or sunscreen compositions. The sunscreen holds the sunscreen active ingredient in place on the skin in order to provide protection from ultraviolet radiation from the sun. The sunscreen resists removal from skin by perspiration and/or immersion in water. An advantage of the sunscreen according to the invention is that certain components that are commonly used in sunscreens can be avoided or the amounts of the components can be reduced. For example, the sunscreen according to the invention can be provided without or with reduced amounts of alcohols, silicones, and surfactants compared with many commercially available sunscreens.

Skin Bonding Polymer Composition

The sunscreen can include a skin bonding polymer composition. The skin bonding polymer composition can be any polymer composition that, when applied to skin, bonds to the skin and holds the sunscreen active ingredient in place on the skin. The skin bonding polymer composition includes a hydrophobic polymer/hydrophilic polymer adduct and can include water and other components. Skin bonding polymer compositions that can be used according to the invention include the topical compositions disclosed in U.S. application Ser. No. 09/933,275 that was filed with the U.S. Patent and Trademark Office on Aug. 20, 2001, and U.S. Patent Publication No. US-2002-0051797-A1. The entire disclosures of U.S. application Ser. No. 09/933,275 and U.S. Patent Publication No. US-2002-0051797-A1 are incorporated herein by reference.

The sunscreen can bind or adhere to skin tissue for a length of time and can hold or contain sunscreen active ingredients within the composition. It is expected that the sunscreen is able to adhere or bind to skin tissue for at least about four hours and hold the sunscreen active ingredients contained therein in proximity to skin tissue for that length of time. In general, it is expected that the sunscreen will bind or adhere to skin tissue for at least four hours even after several applications of washing and scrubbing of the skin tissue. It is expected that the natural exfoliation of the skin will cause the removal of most of the sunscreen from the skin tissue.

The skin bonding polymer composition can be prepared from a topical composition precursor that can be prepared by melt processing a hydrophobic polymer composition and a hydrophilic polymer composition to provide an interaction between the hydrophobic polymer composition and the hydrophilic polymer composition. It should be understood that the phrase “melt processing” refers to mixing the hydrophobic polymer composition and the hydrophilic polymer composition under conditions that provide that the hydrophobic polymer component of the hydrophobic polymer composition and the hydrophilic polymer component of the hydrophilic polymer composition are in a liquid state so that they sufficiently mix. When the polymers are sufficiently mixed, it is believed that an interaction forms between the hydrophobic polymer component and the hydrophilic polymer component. The melt processing temperature can be at least about 50° C. and can be at least about 125° C. to generate this interaction.

It is believed the interaction exhibited between the hydrophobic polymer component and the hydrophilic polymer component is a type of complex formation reaction, and that the complexes, once formed, are stable in water at temperatures up to 65° C. and at a pH range of 3.0 to 9.0. By stable, it is meant that the complexes do not favor disassociation. It is believed that this interaction provides the sunscreen with an ability to bind or hold onto sunscreen active ingredients that may be hydrophobic and that can be emulsified in water, and provides the sunscreen with an ability to bind to skin and/or substrates of predominantly hydrophobic character. The result of the interaction between the hydrophobic polymer component and the hydrophilic polymer component can be referred to as a hydrophobic polymer/hydrophilic polymer adduct. It should be understood that the term “adduct” is used to refer to the interaction between the hydrophobic polymer component and the hydrophilic polymer component. The interaction may be a form of complexing, but that is only theory. Accordingly, it should be understood that the term “adduct” is not meant to limit the polymer component to a particular theory of interaction.

It is believed that the interaction between the hydrophobic polymer component and the hydrophilic polymer component can be achieved more easily in the absence of water. It is expected that that if the hydrophilic polymer component becomes dissolved in water before forming the complex, it will become more difficult to sufficiently mix the hydrophobic polymer component and the hydrophilic polymer component to provide the desired level of interaction. Although a convenient technique for providing the desired level of interaction between the hydrophobic polymer component and the hydrophilic polymer component is melt mixing, it is expected that other techniques can be used to achieve the desired level of interaction.

Hydrophobic Polymer Composition

The hydrophobic polymer composition can include components having repeating pyrrolidone/alkylene groups. Exemplary polymers having repeating pyrrolidone/alkylene groups include poly(vinylpyrrolidone/alkylene) polymers. Poly(vinylpyrrolidone/alkylene) polymers include those polymers obtained by polymerizing alkylene substituted vinylpyrrolidone. Poly(vinylpyrrolidone/alkylene) polymers can be represented by the following general formula:

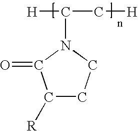

wherein R represents a carbon chain substitute such as an alkylene group and n represents the number of repeating units. The R group is preferably sufficiently long so that the polymer remains relatively water insoluble and should not be too long so that the polymer is difficult to melt process. The alkylene group can contain a length of at least about 10 carbon atoms and can contain no more than about 25 carbon atoms. The alkylene group can contain between about 14 carbon atoms and about 22 carbon atoms, and can contain between about 15 carbon atoms and about 19 carbon atoms.

The poly(vinylpyrrolidone/alkylene) polymers that can be used according to the invention can have a molecular weight that is sufficiently high so that the polymer maintains its water insolubility but the molecular weight should not be so high that it becomes difficult to melt process the polymer. The weight average molecular weight of the poly(vinylpyrrolidone/alkylene) polymer can be between about 3,000 and about 400,000. Another way to characterize the size of the poly(vinylpyrrolidone/alkylene) polymer is by the number of repeating units (n). In the case of a poly(vinylpyrrolidone/alkylene) polymer having a weight average molecular weight of between about 6,000 and about 30,000, the poly(vinylpyrrolidone/alkylene) polymer can have between about 20 and about 80 repeating units, and can have between about 30 and about 50 repeating units. It should be understood that repeating units refer to the residues of vinylpyrrolidone/alkylene groups.

Exemplary poly(vinylpyrrolidone/alkylene) polymers that can be used according to the invention include poly(vinylpyrrolidone/1-eicosene) and poly(vinylpyrrolidone/hexadecene). Poly(vinylpyrrolidone/1-eicosene) can be referred to as PVPE and is commonly used in pharmaceutical and cosmetic preparations. An exemplary form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. An exemplary form of PVPH is available as a viscous yellow liquid that is insoluble in water and has a low oral toxicity (LD₅₀>64000 mg/kg), has about 39 to 40 repeating units, a molecular weight of about 14,000, and exhibits no demonstrable dermal toxicity.

PVPE and PVPH differ in the length of the hydrocarbon side chain, and are used extensively in the skin care industry, usually in concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy.

The hydrophobic polymer composition used according to the invention can be provided as a mixture of different poly(vinylpyrrolidone/alkylene) polymers. The mixture of different poly(vinylpyrrolidone/alkylene) polymers can include at least 5 wt. % of a first poly(vinylpyrrolidone/alkylene) polymer based on the weight of the hydrophobic polymer composition. The hydrophobic polymer composition can include between about 5 wt. % and about 54 wt. % of the first poly(vinylpyrrolidone/alkylene) polymer. The second poly(vinylpyrrolidone/alkylene) polymer can be provided in an amount of at least about 46 wt. % and can be in a range of between about 46 wt. % and 95 wt. % based on the weight of the hydrophobic polymer composition. For a hydrophobic polymer composition containing a first poly(vinylpyrrolidone/alkylene) polymer and a second poly(vinylpyrrolidone/alkylene) polymer, the mole ratio of the first polymer to the second polymer can be between about 1:22 and about 1:1. When the hydrophobic polymer composition contains a mixture of different poly(vinylpyrrolidone/alkylene) polymers, the poly(vinylpyrrolidone/alkylene) polymers can be selected to provide improved properties compared to a sunscreen having a hydrophobic polymer composition containing a single poly(vinylpyrrolidone/alkylene) polymer.

When the hydrophobic polymer composition is provided as a mixture of PVPH and PVPE, the PVPH can be provided in a range of between about 46 wt. % to about 95 wt. % and the PVPE can be provided in a range of between about 5 wt. % and about 65 wt. %, based upon the weight of the hydrophobic polymer composition.

Hydrophilic Polymer Composition

The hydrophilic polymer composition that can be used according to the invention includes at least one hydrophilic polymer and may include a mixture of hydrophilic polymers. The hydrophilic polymers that can be used according to the invention include polymers having repeating carboxylic acid groups and/or hydroxyl groups. Exemplary hydrophilic polymers that can be used according to the invention include polyacrylic acid polymers and poly(maleic acid/methylvinylether) copolymers. The hydrophilic polymers should have a molecular weight that is not too high so that the hydrophilic polymer becomes difficult to process.

Polyacrylic acid polymers that can be used according to the invention include those having a weight average molecular weight of at least about 50,000. Polyacrylic acid polymers that can be used include those having a weight average molecular weight between about 50,000 and about 4,000,000. The polyacrylic acid polymers can have a level of cross-linking that is less than about 1%. A general structural representation of polyacrylic acid polymers is shown below:

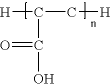

wherein n is the number of repeating units and can be between about 1,000 and about 20,000.

Poly(maleic acid/methylvinylether) copolymers that can be used according to the invention can have a weight average molecular weight of at least about 50,000, and can have a weight average molecular weight of between about 50,000 and about 4,000,000. The weight average molecular weight can be between about 70,000 and 2,500,000. A general structural representation of poly(maleic acid/methylvinylether) copolymers is shown below:

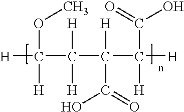

wherein n is the number of repeating units and can be between about 200 and about 20,000.

Additional hydrophilic polymers that can be used according to the invention include starch, derivatives of starch, polyvinyl alcohol, cellulose, derivatives of cellulose, carboxymethyl cellulose, cyclodextrins, and dextrans. The weight average molecular weight of the hydrophilic polymers is preferably sufficient to provide solubility in water but not too high to become difficult to process. Exemplary starches include amylopectin and polyglucose. Starches that can be used according to the invention can have a weight average molecular weight of between about 50,000 and about 20,000,000. A derivative of starch that can be used according to the invention includes partially hydrolized starch. Cellulose that can be used according to the invention can have a weight average molecular weight of between about 50,000 and about 15,000,000. Polyglucose that can be used according to the invention can be characterized as low fraction polyglucose having a weight average molecular weight of between about 60,000 and about 90,000, and high fraction polyglucose having a weight average molecular weight of between about 90,000 and about 300,000. An exemplary low fraction polyglucose material that can be used according to the invention is available under the name Dextran-70. In general, this type of polyglucose has all alpha 1-6 linkages. Starch derivatives that can be used according to the invention include those starch derivatives having alpha 1-4 linkages. An example of this type of starch derivative includes cyclodextrins. Exemplary cyclodextrins that can be used according to the invention include those that act to provide a cavity within the molecule large enough to contain components desirable for topical applications. Cyclodextrins that can be used according to the invention can have a molecular weight of between about 900 and about 1,400. Polyvinyl alcohols that can be used according to the invention include those with a weight average molecular weight of between about 50,000 and about 200,000.

Exemplary hydrophilic polymers that can be used according to the invention include those polymers having the following melting temperature range and the following maximum temperature range beyond which it is expected decomposition of the polymer will occur. Exemplary poly(maleic acid/methylvinylether) copolymers that can be used include those having a melting temperature range of between about 60° C. and about 65° C. and a maximum temperature range of between about 80° C. and about 90° C. Exemplary polyacrylic acid polymers that can be used include those having a melting temperature range of between about 65° C. and about 70° C. and a maximum temperature range of between about 80° C. and about 90° C. Exemplary carboxymethyl cellulose polymers that can be used include those having a melting temperature range of between about 55° C. and about 60° C. and a maximum temperature range of between about 75° C. and about 80° C. Exemplary polyvinyl alcohol polymers that can be used include those having a melting temperature range of between about 50° C. and about 55° C. and a maximum temperature range of between about 65° C. and about 70° C. Exemplary starches that can be used include those having a melting temperature range of between about 40° C. and about 45° C. and a maximum temperature 10 range of between about 50° C. and about 55° C. Exemplary dextrans that can be used include those having a melting temperature range of between about 37° C. and about 40° C. and a maximum temperature range of between about 45° C. and about 50° C. Exemplary β-cyclodextrins that can be used according to the invention include those having a melting temperature range of between about 40° C. and about 45° C. and a maximum temperature range of between about 65° C. and about 70° C.

The hydrophobic polymer composition and the hydrophilic polymer composition can be combined and heated to at least about 50° C. to provide a polymer melt. The composition can be heated to at least about 125° C. under mixing to form complexes between the hydrophobic and hydrophilic polymers. It should be understood that a polymer melt refers to a polymer that flows or becomes a liquid when heated and is not meant to refer to a polymer that forms a liquid as a result of being dissolved in a solvent.

The complex formation step can be carried out in a relatively anhydrous environment. That is, the amount of water provided in the composition during the complex formation step can be less than about 1 wt. %. Once the desired level of complex formation has occurred, the composition can be hydrated with water.

The hydrophobic polymer composition and the hydrophilic polymer composition can be mixed together in amounts sufficient to provide a ratio of pyrrolidone groups to the combination of carboxylic acid groups and hydroxyl groups of between about 1:1 and about 5:1. The ratio of the structures causing the observed interaction between the hydrophobic polymer composition and the hydrophilic polymer composition can be referred to as “functional group parity.” The ratio of pyrrolidone groups to the combination of carboxylic acid groups and hydroxyl groups can be between about 1.5:1 and about 3:1. In order to drive the complex formation reaction, it is desirable to provide an imbalance between the two types of groups. Accordingly, it is generally desirable to provide more of the pyrrolidone groups than the combination of carboxylic groups and the hydroxyl groups. It should be understood that the reference to a “combination of carboxylic groups and hydroxyl groups” refers to the total amount of carboxylic groups and hydroxyl groups present but does not require the presence of both carboxylic groups and hydroxyl groups. For example, the value of the combination of carboxylic groups and hydroxyl groups can be determined for a composition that contains only carboxylic groups. Similarly, the value can be determined for a composition that contains only hydroxyl groups.

During the complex formation step, the amounts of hydrophobic polymer composition and hydrophilic polymer composition can be characterized on a weight percent basis. For example, about 2 wt. % to about 28 wt. % hydrophilic polymer composition and about 72 wt. % to about 98 wt. % hydrophobic polymer composition can be combined to provide for complex formation. About 8 wt. % to about 25 wt. % hydrophilic polymer composition and about 72 wt. % to about 95 wt. % hydrophobic polymer composition can be combined to form the complex. During the complex formation step, the amount of water available in the composition is preferably less than about 1 wt. %. Although the complex forming composition can be relatively anhydrous, it is expected that the amount of water will be between about 0.3 wt. % and about 1.0 wt. %.

Once the hydrophobic polymers and the hydrophilic polymers have sufficiently reacted or interacted to form a complex, water can be added to the composition to provide a stable aqueous composition that can be relatively easily further hydrated. The stable aqueous composition that can be easily diluted further with water can be referred to as the concentrate. It is generally desirable to hydrate the composition to a water content that provides a relatively stable composition and that allows for water to be added at a later date without much difficulty. Although water can be added to the composition to a level equivalent to the level of the sunscreen use solution, it is desirable to minimize the amount of water to avoid having to ship water. Shipping excess water is expected to add cost to the composition. In addition, it has been found that the first hydration of the topical composition precursor is the most difficult hydration step because of the need to control the conditions of hydration. After the first hydration to a water content of at least about 30 wt. %, it is expected that further hydrations to higher water contents are relatively easy and can be accomplished by simply mixing the composition with water. Accordingly, the amount of water provided in the composition when made available as a concentrate for shipment is preferably between about 30 wt. % and about 45 wt. %. When the composition includes about 30 wt. % to about 45 wt. % water, it is expected that the composition will include between about 3 wt. % and about 10 wt. % hydrophilic polymer composition and between about 30 wt. % and about 50 wt. % hydrophobic polymer composition.

Water can be added to the relatively anhydrous composition by mixing water and the relatively anhydrous composition at a temperature and for a time sufficient to allow the composition to become hydrated without losing significant amounts of interaction between the hydrophobic polymer composition and the hydrophilic polymer composition. In general, the relatively anhydrous composition is hydrated by heating to at least 60° C. and adding water while mixing. Preferably, the composition is heated to at least about 65° C. and more preferably at least about 70° C. A preferred temperature range is about 65° C. to about 80° C.

The relatively anhydrous composition can be referred to as the topical composition precursor and generally refers to the hydrophobic polymer/hydrophilic polymer adduct. The skin bonding polymer composition can refer to a composition that contains only the hydrophobic polymer/hydrophilic polymer adduct, and it can refer to a composition wherein the hydrophobic polymer/hydrophilic polymer adduct is diluted with water. In general, it is desirable to have a sufficient amount of water in the skin bonding polymer composition that allows one to formulate the skin bonding polymer composition into a sunscreen according to the invention. If there is too little water in the skin bonding polymer composition, it may become difficult to formulate the sunscreen, and if there is too much water in the skin bonding polymer composition, it is possible that the skin bonding polymer composition will phase separate. In general, it is desirable to have the skin bonding polymer composition as a single phase to facilitate formulating the sunscreen. It is expected that the skin bonding polymer composition can contain water in an amount of up to about 95 wt. %. The skin bonding polymer composition can be referred to as the concentrate. It is expected that the skin bonding polymer composition will have a water concentration of between about 30 wt. % and about 45 wt. %. In addition, it is expected that formulators will either further hydrate the skin bonding polymer composition or use it as it is made available to them.

Additional components can be added to the skin bonding polymer composition. For example, it may be desirable to add a component that helps stabilize the hydrophobic polymer/hydrophilic polymer adduct, and to help preserve and/or maintain the composition.

Sunscreen Active Ingredients

The sunscreen composition according to the invention can contain a sunscreening effective amount of a sunscreen active ingredient. Exemplary sunscreen active ingredients include one or more UV-A actives, UV-B actives, and mixtures of UV-A actives and UV-B actives. UV-A type sunscreen actives protect against long wavelength actinic radiation of the sun in the 320 to 400 nm range and UV-B type sunscreen actives protect against shorter wavelength, actinic radiation of the sun in the 290-320 nm range. The sunscreen active ingredients can be referred to more simply as the sunscreen actives.

The sunscreen includes a sufficient amount of the sunscreen actives to provide the desired SPF value. SPF values refer to those values obtained with methods based upon the test procedures proposed to the Title 21 of the United States Code of Federal Regulations, §§ 352.72, pages 27690 through 27693 (final monograph).

Sunscreen actives that can be used according to the invention include any of the sunscreen actives that provide desired protection from the sun's radiation and that are approved for use in a composition that contacts skin tissue. Exemplary sunscreen actives include trade name of para-aminobenzoic acid (PABA) up to about 15 weight percent or from about 5 to 15 weight percent in admixture with other sunscreen actives; cinoxate up to about 3 weight percent or about 1 to 3 weight percent in admixture; diethanoloamine methoxycinnamate up to 10 weight percent or about 8 to 10 weight percent in admixture; digalloyl trioleate up to 5 weight percent or about 2 to 5 weight percent in admixture; dioxybenzone up to 3 weight percent alone or in admixture; ethyl 4[bis(hydroxypropyl)]aminobenzoate up to 5 weight percent or about 1 to 5 weight percent in admixture; glyceryl aminobenzoate up to 3 weight percent or about 2 to 3 weight percent in admixture; homosalate up to 15 weight percent or about 4 to 15 weight percent in admixture; lawsone up to 0.25 weight percent with dihydroxyacetone up to 3 weight percent; menthyl anthanilate up to 5 weight percent or about 3.5 to 5 weight percent in admixture; octocrylene up to 10 weight percent or 7 to about 10 weight percent in admixture; octyl methoxycinnamate up to 7.5 weight percent or about 2 to 7.5 weight percent in admixture; octyl salicylate up to 5 weight percent or about 3 to 5 weight percent in admixture; oxybenzone up to 6 weight percent or about 2 to 6 weight percent in admixture; padimate “O” up to 8 weight percent or about 1.4 to 8 weight percent in admixture; phenylbenzimidazole sulfonic acid up to 4 weight percent or about 1 to about 4 weight percent admixture; red veterinary petrolatum up to 95 percent or about 30 to 95 weight percent in admixture; sulisobenzone up to 10 weight percent or about 5 to 10 weight percent in admixture; titanium dioxide up to 25 weight percent or about 2 to 25 weight percent in admixture; trolamine salicylate up to 12 weight percent or about 5 to 12 weight percent in admixture; avobenzone up to 3 weight percent; and zinc oxide up to 25 weight percent.

Typical suitable UV-B type sunscreening actives include benzophenone-3, benzophenone-8, substituted para-aminobenzoates, e.g. alkyl esters of para-methoxycinnamte, octyl methoxycinnamte and octyl para-methoxycinnamate, available from Givaudan Corp., Clifton, N.J. 07104 under the tradename Parsol MCX and usually present in the range of about 2 to 7.5 weight percent or octyl slicylate available from Harmann and Riemer, Springfield, N.J. 07081, usually in the range of about 3 to 5 weight percent. Typical suitable UV-A type sunscreening actives include oxybenzone, usually in the range of about 2 to about 6 weight percent. Except as noted otherwise, one or more sunscreen actives can be employed in the present composition in amounts up to 35 weight percent, in amounts of between about 6 to about 30 weight percent, and in amounts between about 10 to about 25 weight percent, based on the weight of the sunscreen.

Water

The sunscreen includes water in an amount sufficient to provide a composition that can be conveniently applied to skin. If the sunscreen includes too little water, it is expected that the sunscreen will be difficult to apply to skin. If the sunscreen includes too much water, it is expected that the sunscreen will lose sunscreening ability. In general, it is expected that the sunscreen composition will contain at least about 40 wt. % water, and may include up to about 80 wt. % water. In general, it is expected that the sunscreen composition will include between about 50 wt. % and about 75 wt. % water. It should be understood that the water component, when identified as a component different from the skin bonding polymer composition, is not meant to restrict the amount of water provided in the skin bonding polymer composition.

Additional Components

The sunscreen can include additional components including thickeners, preservatives, humectants, skin healing agents, pH neutralizers, emollients, and vitamins. It should be understood that these components are optional and need not be incorporated into the sunscreen composition. Additional components often found in skin lotion compositions can be included in the sunscreen composition. The additional components include vitamins, α-hydroxy acids, surfactants, pigments, and dyes. Specific examples of components that can be included in the sunscreen are identified in CTFA International Cosmetic Ingredient Dictionary & Handbook, 9th Edition, Cosmetic, Toiletry, and Fragrance Association, 2002.

Thickeners that can be incorporated into the sunscreen include those components that thicken or increase the viscosity of sunscreen so that the sunscreen can be readily applied to skin. Thickeners that can be used in the sunscreen include those components often referred to as viscosity controlling agents.

Exemplary thickeners or viscosity controlling agents that can be provided in the sunscreen include alkane triols; acrylates; substituted celluloses; gums, natural and/or synthetic; long chain alcohols including those having about 9 to about 24 carbon atoms; polyglycols including polyethylene glycols, polypropylene glycols, polybutylene glycols, polyethylene propylene glycols, and mixtures thereof; waxes, natural and/or synthetic; polyquaternary compounds; hydrogenated oils; glycol esters; fatty acid esters; long chain acids; acid amides; silicate sales; polyamides; substituted ammonia; and mixtures thereof.

The sunscreen may or may not include a thickener. In general, it is expected that if the sunscreen is intended to be applied by spray application, the sunscreen may forego the use of a thickener. The use of a thickener may be desired when the sunscreen is to be applied by hand and rubbed onto the skin. When the sunscreen includes a thickener, the thickener can be provided in an amount that provides the desired level of thickening. An exemplary range of thickener is between about 0.1 wt. % and about 2 wt. %, and between about 0.2 wt. % and about 0.4 wt. %, based on the weight of the sunscreen.

The amounts of sunscreen actives should be sufficient to provide the sunscreen with an SPF of at least 2, and more preferably at least 10. In general, it is expected that most desired sunscreens will have an SPF of between about 10 and about 40, and can have SPF values between about 15 and about 30.

The sunscreen can include preservatives for prevention of bacterial, fungal, and/or yeast contamination. Exemplary preservatives that can be used in the sunscreen include benzoic acid, derivatives and salts of benzoic acid, parbens, oxazolidines, quaternary amines, derivatives and salts of quaternary amines, chlorinated aromatic compounds and phenols, hydantoins, cresols and derivatives, imiazolindinyl urea, iodopropynyl butylcarbamate, sulfites, bisulfites, iodates, and aldehydes. The sunscreen can include any of the preservatives commonly used or known to be suitable for topically applied compositions.

The sunscreen can be formulated without a preservative. It is expected that the preservative will increase the shelf life of the sunscreen by reducing or preventing the growth of bacteria, fungus, and/or yeast. When the sunscreen includes a preservative, the preservative is preferably provided in an amount sufficient to provide a desired level of protection from growth of bacteria, fungus, and/or yeast. In general, for most preservatives, it is expected that the amount of preservative will be provided at a level of between about 0.25 wt. % and about 0.5 wt. %, and can be provided at a level of between about 0.3 wt. % and about 0.4 wt. %, based on the weight of the sunscreen.

Humectants that can be included in the sunscreen include those commonly used or suitable for use in topical compositions. Exemplary humectants include glycerols, glycerin, glycerates, aliphatic alcohols, aliphatic polyols, silicones, silicone copolyols, amino acids, salts and derivatives of amino acids, pyrrolidone carboxylic acid, salts and derivatives of pyrrolidone carboxylic acid, glycols, polyglycols, sugars, acid derivatives and salts, protein hydrolysates, and salts and derivatives of protein hydrolysates.

The sunscreen can include a humectant when it is desirable to provide the sunscreen with a moisturizing feel or effect. The humectant is an optional component and can be excluded from the sunscreen. When the sunscreen includes a humectant, it can be included in an amount of between about 0.25 wt. % and about 7 wt. %, and can be provided in an amount of between about 0.5 wt. % and about 2 wt. %, based on the weight of the sunscreen.

Skin healing agents that can be included in the sunscreen include those components commonly used or capable of use in topical compositions. Exemplary skin healing agents include aloe vera and other plant extracts, vitamin A, vitamin D, and vitamin D2.

The sunscreen can include a skin healing agent when it is desirable to provide the properties associated with a skin healing agent. It should be understood that the sunscreen can be provided without a skin healing agent. When the sunscreen includes a skin healing agent, it can be included in an amount that provides for skin healing properties. In general, it is expected that the skin healing agent can be provided in an amount of between about 0.02 wt. % and about 1 wt. %, and can be provided in an amount of between about 0.03 wt. % and about 0.1 wt. %, based on the weight of the sunscreen.

The sunscreen can include a pH adjusting agent to provide the sunscreen with a relatively neutral pH. In general, it is desirable to provide a sunscreen at a pH that will not damage or irritate skin tissue. Accordingly, it is generally desirable to provide the sunscreen composition with a pH of between about 4 and about 8 and preferably a pH of about 7. Exemplary pH adjusting agents include buffering agents such as carbonates, phosphates, amines, acids, citrates, and mixtures thereof.

The sunscreen does not need to include a pH adjusting agent if the pH of the sunscreen is at a desired level. When the pH adjusting agent is included, it is preferably provided in an amount to provide the sunscreen with a pH of between about 4 and about 8. In general, it is expected that when the sunscreen includes a pH adjusting agent, it is included in an amount of between about 0.1 wt. % and about 2 wt. %, and can be provided in an amount of between about 0.2 wt. % and about 1 wt. %, based on the weight of the sunscreen.

An emollient can be included in the sunscreen to provide the sunscreen with a desirable feel or texture. Emollients that can be used in the sunscreen include those commonly used or capable of use in topical compositions. Exemplary emollients include esterified oils, hydrogenated oils, esterified sugars, waxes, oils, long chain fatty acids, salts and alcohols (C₈-C₄₀), polyacrylates, esters and salts of polyacrylates, glycols, polyglycols, esters of glycols, esters of polyglycols, silicones, copolyol silicones, amides, polyamides, amines, polyamines, polyamine amides, salts of amides, salts of polyamines, salts of amines, salts of polyamine amides, starches, cellulose, derivatives of starches, derivatives of cellulose, polymethacrylates, copolymers, salts and esters of copolymers, nylon derivatives, vinyl polymers, salts of sulphate, phosphates, borates, silicates, proteins, and substituted proteins.

The sunscreen can be provided without an emollient. When the emollient is included in the sunscreen, it is preferably provided in an amount that provides the sunscreen with a desirable feel or texture. In most applications, it is expected that the amount of emollient in the sunscreen will be between about 1 wt. % and about 4 wt. %, and can be provided in an amount of between about 2 wt. % and about 3 wt. %, based on the weight of the sunscreen.

The sunscreen can include anti-oxidants to help increase the shelf life of the sunscreen by reducing oxidation of the sunscreen actives. Exemplary anti-oxidants that can be used include vitamins such as vitamin E, vitamin C, vitamin A, and vitamin D, and derivatives thereof. Exemplary anti-oxidants include α-tocopherols which can be characterized as natural or synthetic Vitamin E.

The sunscreen can be formulated without an anti-oxidant. When the sunscreen includes an anti-oxidant, the anti-oxidant is preferably provided in an amount that provides anti-oxidant properties in the sunscreen. In general, it is expect that the anti-oxidant can be provided in an amount of between about 0.2 wt. % and about 2 wt. %, and can be provided in an amount of between about 0.7 wt. % and about 1.5 wt. %, based on the weight of the sunscreen.

The sunscreen according to the invention can exclude components found in many commercially available sunscreens, or can reduce the amount of certain components found in commercially available sunscreens. For example, many commercially available sunscreens include solvents, surfactants, and silicones. It is believed that certain commercially available sunscreens include a solvent such as ethyl alcohol in a relatively large amount (such as between about 50 wt. % and about 75 wt. %) when the sunscreen is intended to be applied by spray application. In addition, it is believed that many commercially available sunscreens include silicones in an amount of between about 3 wt. % and about 12 wt. % in order to adhere sunscreen actives to the skin. It is believed that surfactants are often used to help solubilize the silicones in the sunscreen. For example, surfactants such as glycerol stearates and polyoxyethylene esters can be used in amounts of between about 1 wt. % and about 2 wt. % to help solubilize the silicones. It is understood that the surfactants can be used or characterized as emulsifiers. It is believed that the high level of solvent used in certain sunscreens can have a deleterious effect on skin by causing drying of the skin. In addition, it is believed that the surfactants help to reduce the water resistance of many commercially available sunscreens.

The sunscreen according to the invention can exclude or reduce the concentration of many of the components commonly used in cosmetics and sunscreens. For example, the sunscreen according to the invention can be formulated with less than 5 wt. % solvent (organic solvent) such as ethyl alcohol, less than about 0.5 wt. % of surfactant or emulsifier such as glycerol stearates and polyoxyethylene esters, and less than about 1 wt. % silicones. The sunscreen can be formulated with less than 1 wt. % organic solvent such as an alcohol. In general, it is expected that the sunscreen according to the invention can be provided without any organic solvent, surfactant, and/or silicone.

Table 1 identifies an exemplary sunscreen compositions having SPF 15-30.

TABLE 1
SPF 15-30 Sunscreen

| Component | Range (wt. %) | Range (wt. %) |
|---|---|---|
| water | 40-80 | 50-75 |
| skin bonding polymer composition | 6-15 | 8-12 |
| sunscreen active ingredient | 6-30 | 10-25 |
| thickener | 0.1-2 | 0.2-0.4 |
| preservative | 0.25-0.5 | 0.3-0.4 |
| humectant | 0.25-7 | 0.5-2 |
| skin healing agent | 0.02-1 | 0.03-0.1 |
| pH adjusting agent | 0.1-2 | 0.2-1 |
| emollient | 1-4 | 2-3 |
| anti-oxidant | 0.2-2 | 0.7-1.5 |

A sunscreen having SPF 30 can have the formulation identified in Table 2.

TABLE 2
SPF 30 Sunscreen

| Components | % by wt. |
|---|---|
| water | 61.82 |
| carbomer | 0.25 |
| preservative | 0.50 |
| glycerin | 3.00 |
| aloe vera 100:1 powder | 0.03 |
| triethanolamine | 0.90 |
| octyl methoxycinnamate | 7.50 |
| oxybenzone | 6.00 |
| octyl salicylate | 5.00 |
| octocrylene | 4.00 |
| coco butter | 2.00 |
| α-tocopherols | 1.00 |
| skin bonding polymer composition | 8.00 |

The carbomer component is a thickener available under the name Carbopol Ultra Z10.

The identified preservative is available under the name Germall.

The sunscreen actives include octyl methoxycinnamate, oxybenzone, octyl salicylate, and octocrylene.

Cocoa butter is an emollient.

α-tocopherols is an antioxidant and is generally referred to as a synthetic form of Vitamin E.

A sunscreen having SPF 15 is identified in Table 3.

TABLE 3
SPF 15 Sunscreen

| Components | % by wt. |
|---|---|
| water | 77.25 |
| Carbomer | 0.25 |
| 2-phenoxyethanol | 0.50 |
| glycerin | 2.00 |
| aloe | 0.10 |
| triethanolamine | 0.90 |
| octyl methoxycinnamate | 3.00 |
| oxybenzone | 3.00 |
| octyl salicylate | 2.00 |
| octocrylene | 2.00 |
| α-tocopherols | 1.00 |
| skin bonding polymer composition | 8.00 |

The skin bonding polymer compositions identified in Tables 2 and 3 can be any of the exemplary polymer compositions identified in Tables 4-11.

TABLE 4

| Components | % by wt. |
|---|---|
| water | 40.58 |
| maleic acid/methylvinylether copolymer | 6.86 |
| polyvinylpyrrolidone 1-hexadecene | 46.46 |
| polyvinylpyrrolidone 1-eicosene | 5.60 |
| 2-phenoxyethanol | 0.50 |

TABLE 5

| Components | % by wt. |
|---|---|
| water | 41.15% |
| starch* | 7.35% |
| polyvinylpyrrolidone 1-hexadecene | 23.46% |
| polyvinylpyrrolidone 1-eicosene | 27.54% |
| phenoxyethanol | 0.5% |

*Soluble starch from Cerestar.

TABLE 6

| Components | % by wt. |
|---|---|
| water | 45.5% |
| starch* | 4.0% |
| polyvinylpyrrolidone 1-hexadecene | 45.0% |
| polyvinylpyrrolidone 1-eicosene | 5.0% |
| phenoxyethanol | 0.5% |

*Soluble starch from Cerestar.

TABLE 7

| Components | % by wt. |
|---|---|
| water | 38.17% |
| carboxymethyl cellulose | 6.83% |
| polyvinylpyrrolidone 1-hexadecene | 25.00% |
| polyvinylpyrrolidone 1-eicosene | 29.50% |
| phenoxyethanol | 0.5% |

TABLE 8

| Components | % by wt. |
|---|---|
| water | 45.5% |
| carboxymethyl cellulose | 4.0% |
| polyvinylpyrrolidone 1-hexadecene | 45.0% |
| polyvinylpyrrolidone 1-eicosene | 5.0% |
| phenoxyethanol | 0.5% |

TABLE 9

| Components | % by wt. |
|---|---|
| water | 39.0% |
| polyglucose* | 5.5% |
| polyvinylpyrrolidone 1-hexadecene | 50.0% |
| polyvinylpyrrolidone 1-eicosene | 5.0% |
| phenoxyethanol | 0.5% |

*Dextran-70.

TABLE 10

| Components | % by wt. |
|---|---|
| water | 30.0% |
| polyacrylic acid* | 6.0% |
| polyvinylpyrrolidone 1-hexadecene | 58.0% |
| polyvinylpyrrolidone 1-eicosene | 5.5% |
| phenoxyethanol | 0.5% |

*Carbopol Ultrez-10 from BF Goodrich.

TABLE 11

| Components | % by wt. |
|---|---|
| water | 46.50% |
| polyvinyl alcohol | 3.00% |
| polyvinylpyrrolidone 1-hexadecene | 45.00% |
| polyvinylpyrrolidone 1-eicosene | 5.00% |
| phenoxyethanol | 0.5% |

An exemplary sunscreen composition having SPF 15 is identified in Table 12. It should be understood that the skin bonding polymer composition can be any of the skin bonding polymer compositions identified in Tables 4-11.

The following examples were carried out in order to demonstrate certain teachings of the invention. It should be understood that the invention is not limited to the examples of this application.

### EXAMPLE

### Evaluation of the Sun Protection Factor (SPF) of a Sunscreen Formula Under Very Water Resistant Conditions

The objective of this experiment is to measure the SPF of a sunscreen in 20 qualified human volunteers under “very water resistant” conditions, according to the FDA final monograph. The sunscreen tested is the sunscreen reported in Table 2.

On the first day of the study each subject received a series of UV doses from a xenon arc solar simulator to an unprotected site on the mid-back. On the second day the minimal erythema dose (MED) was determined as the lowest UV dose which produced mild erythema reaching the borders of the exposed site. Then 100 mg of the test product was applied to a 50 cm² area of the mid-back. After a 15-minute drying period, subjects sat in a tub with moving water for 4 periods of 20 minutes of water immersion, separated by 20 minutes out of the water.

After drying for 15 minutes, 100 mg of the 8% homosalate standard sunscreen, which has an expected SPF of 4.47, was applied to a 50 cm² area adjacent to the test product, then UV doses as described in the protocol were administered to the respective sunscreen-protected areas. A series of UV doses was also administered to a second unprotected site.

On the third day the MED was determined for the sunscreen-protected sites and the unprotected site and the SPF's of each sunscreen were calculated as the ratio of the MED's for each protected site to the MED for the unprotected site.

SPF values for the test products are listed in Table 12.

A total of 23 subjects, who provided written informed consent, were enrolled in the study. Subjects 07 and 08 had no MED response after the Day 1 exposures and were disqualified. The 21 subjects who completed the study included 12 men and 9 women, with a mean age was 29.4 (SD=6.9). Subjects included 8 with skin type I, 7 with skin type II, and 6 with skin type III. Subject demographic data are listed in Table 12.

The labeled SPF of a “Very Water Resistant” sunscreen product is the largest whole number that is excluded by a 95% confidence interval for the mean SPF after 80 minutes of water immersion. Additional requirements for acceptance of an SPF test panel are that the mean SPF of the 8% homosalate standard must fall within the standard deviation range of the expected SPF (4.47±1.279) and that the 95% confidence interval for the mean SPF of the standard must contain the value 4.0.

The mean SPF was 31.4 (SD=2.8) and the 95% confidence interval (a) was 1.1, thus the product may be labeled as Very Water Resistant SPF 30.

The mean SPF of the 8% homosalate standard was 4.2 (SD=0.5), which is within 4.47±1.279, and the 95% confidence interval extends from 4.4 to 4.0. Thus the SPF test panel is valid. Subject demographic data and SPF results are listed in Table 12.

TABLE 13
Subject demographic data and SPF results

| | | | | | | Expected SPF 30 | | SPF 4 | | | |
|---|---|---|---|---|---|---|---|---|---|---|---|
| Subject # | Age | Sex | Skin Type | Original MED | Final Med | Med (P) | SPF | MED (P) | SPF | Standard | |
| 01 | 40 | F | II | 8 | 8 | 257 | 32.13 | 40 | 5.00 | Monograph mean | |
| | | | | | | | | | | is 4.47 ± 1.279 | |
| 02 | 26 | F | II | 13 | 13 | 296 | <22.77 | 57 | 4.38 | 4.47 − 1.279 | 3.2 |
| 03 | 38 | F | III | 16 | 13 | 446 | 34.31 | 58 | 4.46 | 4.47 + 1.279 | 5.7 |
| 04 | 27 | F | III | 10 | 13 | 396 | 30.46 | 50 | 3.85 | 3.2 < mean | |
| | | | | | | | | | | (4.2) < 5.7 | |
| 05 | 26 | M | III | 10 | 10 | 321 | 32.10 | 40 | 4.00 | 95% CI | 0.2 |
| 06 | 25 | M | II | 8 | 8 | 223 | 27.88 | 26 | 3.25 | Mean + 95% CI | 4.4 |
| 09 | 42 | M | II | 13 | 13 | 417 | 32.08 | 52 | 4.00 | Mean − 95% CI | 4.0 |

| | | | | | | | | | | |
|---|---|---|---|---|---|---|---|---|---|---|
| 10 | 28 | F | II | 10 | 10 | 321 | 32.10 | 44 | 4.40 | 4.0 < mean |
| | | | | | | | | | | (4.2) < 4.4 |
| 11 | 25 | F | I | 10 | 10 | 300 | 30.00 | 40 | 4.00 | |
| 12 | 32 | M | I | <6 | 6 | 180 | 30.00 | 24 | 4.00 | |
| 13 | 19 | M | I | 8 | 8 | 240 | 30.00 | 35 | 4.38 | |
| 14 | 25 | M | I | 8 | 6 | 182 | 30.33 | 32 | 5.33 | |
| 15 | 29 | F | III | 13 | 13 | 339 | 26.08 | 50 | 3.85 | |
| 16 | 29 | M | I | 8 | 8 | 240 | 30.00 | 35 | 4.38 | |
| 17 | 20 | M | I | 8 | 6 | 240 | 40.00 | 29 | 4.83 | |
| 18 | 45 | F | II | 8 | 8 | 257 | 32.13 | 35 | 4.38 | |
| 19 | 22 | M | I | 8 | 8 | 257 | 32.13 | 32 | 4.00 | |
| 20 | 31 | F | II | 8 | 8 | 240 | 30.00 | 32 | 4.00 | |
| 21 | 33 | M | I | <6 | 5 | 167 | 33.40 | 22 | 4.40 | |
| 22 | 28 | M | III | 13 | 13 | 417 | 32.08 | 52 | 4.00 | |
| 23 | 28 | M | III | 10 | 10 | 300 | 30.00 | 40 | 4.00 | |
| | 29.4 | | | Mean = | 9 | 287 | 31.4 | 39 | 4.2 | |
| | 6.9 | | | SD = | 2.7 | 80.3 | 2.8 | 10.7 | 0.5 | |
| | 21 | | | n = | 21 | 21 | 20 | 21 | 21 | |
| | | | | SE = | 0.6 | t = | 1.7 | | | |
| | | | | SE/Mean = | 0.0 | s = | 2.8 | | | |
| | | | | sqrt(n) = | 4.5 | a = | 1.1 | | | |
| | | | | sqrt(n − 1) = | 4.4 | Mean SPF − a = | 30.3 | | | |

The above specification, examples and data provide a complete description of the manufacture and use of the composition of the invention. Since many embodiments of the invention can be made without departing from the spirit and scope of the invention, the invention resides in the claims hereinafter appended.

In the claims:

1. A sunscreen composition comprising:
(a) a skin bonding polymer composition comprising a hydrophobic polymer/hydrophilic polymer adduct;
(b) at least one sunscreen active ingredient in an amount effective to provide the composition with an SPF value of at least 4; and
(c) water in an amount effective to provide the composition with a texture suitable for application to skin.

2. A sunscreen composition according to claim 1, wherein the hydrophobic polymer/hydrophilic polymer adduct comprises a result of melt mixing:
(i) hydrophobic polymer composition comprising repeating pyrrolidone/alkylene groups, wherein the alkylene groups contain at least 10 carbon atoms; and
(ii) hydrophilic polymer composition comprising repeating carboxylic groups and/or repeating hydroxyl groups.

3. A sunscreen composition according to claim 2, wherein the hydrophobic polymer composition and the hydrophilic polymer composition have a functional group parity provided at a ratio of between about 1:1 and about 5:1.

4. A sunscreen composition according to claim 2, wherein the hydrophobic polymer composition and the hydrophilic polymer composition have a functional group parity provided at a ratio of between about 1.5:1 and about 3:1.

5. A sunscreen composition according to claim 1, wherein the sunscreen active ingredient comprises at least one of aminobenzoic acid, avobenzone, cinoxate, dioxybenzone, homosalate, menthyl anthranilate, octocrylene, octyl methoxycinnamate, octyl salicylate, oxybenzone, padimate O, phenolbenzimidazole sulfonic acid, sulisobenzone, titanium dioxide, trolamine salicylate, zinc oxide, and mixtures thereof.

6. A sunscreen composition according to claim 1, wherein the composition comprises between about 6 wt. % and about 30 wt. % of the at least one sunscreen active ingredient.

7. A sunscreen composition according to claim 1, wherein the water is provided at a concentration of between about 40 wt. % and about 80 wt. % water.

8. A sunscreen composition according to claim 1, wherein the hydrophobic polymer/hydrophilic polymer adduct comprises a result of melt mixing:
(i) a poly(vinylpyrrolidone-alkylene) polymer, wherein the alkylene group contains at least about 10 carbon atoms; and
(ii) at least one of:
(a) polyacrylic acid having a weight average molecular weight of at least about 50,000 and exhibiting less than 1% cross-linking;
(b) poly(maleic acid/methylvinylether) copolymer having a weight average molecular weight of at least about 50,000;
(c) starch;
(d) derivatives of starch;
(e) cellulose;
(f) derivatives of cellulose;
(g) carboxymethyl cellulose;
(h) polyvinyl alcohol;
(i) cyclodextrins;
(j) dextrans; and
(k) mixtures thereof.

9. A sunscreen composition according to claim 1, wherein the skin bonding polymer composition comprises the hydrophobic polymer/hydrophilic polymer adduct and water.

10. A sunscreen composition according to claim 1, wherein the skin bonding polymer composition comprises at least 50 wt. % of the hydrophobic polymer/hydrophilic polymer adduct.

11. A sunscreen composition according to claim 1, further comprising between about 0.1 wt. % and about 2 wt. % of a thickener.

12. A sunscreen composition according to claim 1, further comprising between about 0.25 wt. % and about 0.5 wt. % of a preservative.

13. A sunscreen composition according to claim 1, further comprising between about 0.25 wt. % and about 7 wt. % of a humectant.

14. A sunscreen composition according to claim 1, further comprising between about 0.02 wt. % and about 1 wt. % of a skin healing agent.

15. A sunscreen composition according to claim 1, further comprising between about 0.1 wt. % and about 2 wt. % of a pH adjusting agent.

16. A sunscreen composition according to claim 1, further comprising between about 1 wt. % and about 4 wt. % of an emollient.

17. A sunscreen composition according to claim 1, further comprising between about 0.2 wt. % and about 2 wt. % of an anti-oxidant.

18. A sunscreen composition according to claim 1, wherein the composition comprises less than about 5 wt. % organic solvent, less than about 0.5 wt. % surfactant, and less than about 1 wt. % silicone.

19. A sunscreen composition comprising:
(a) a skin bonding polymer composition comprising a hydrophobic polymer/hydrophilic polymer adduct;
(b) at least one sunscreen ingredient in an amount effective to provide the composition with an SPF value of at least 4;
(c) water in an amount effective to provide the composition with a texture suitable for application to skin;
(d) wherein the sunscreen composition includes less than about 1 wt. % alcohol.

20. A method for manufacturing a sunscreen composition, the method comprising steps of:
(a) mixing a skin bonding polymer composition with at least one sunscreen active ingredient in an amount effective to provide the composition with an SPF value of at least 4 and water in an amount effective to provide the composition with a texture suitable for application to skin to provide the sunscreen composition, wherein the skin bonding polymer composition comprises a hydrophobic polymer/hydrophilic polymer adduct.

21. A method according to claim 20, wherein the hydrophobic polymer/hydrophilic polymer adduct comprises a result of melt processing:
(i) hydrophobic polymer composition comprising repeating pyrrolidone/alkylene groups, wherein the alkylene groups contain at least 10 carbon atoms;
(ii) hydrophilic polymer composition comprising repeating carboxylic acid groups and/or repeating hydroxyl groups.

22. A method according to claim 20, wherein the step of mixing further comprises:
(a) adding to the sunscreen composition at least one of a thickener, preservative, humectant, skin healing agent, pH adjusting agent, emollient, and anti-oxidant.

23. A method according to claim 20, wherein the sunscreen composition is prepared with no more than about 5 wt. % organic solvent, no more than about 0.5 wt. % surfactant, and no more than about 1 wt. % silicone.

24. A method for using a sunscreen composition, the method comprising:
(a) applying a sunscreen composition to skin, the sunscreen composition comprising:
(i) a skin bonding polymer composition comprising a hydrophobic polymer/hydrophilic polymer adduct;
(ii) at least one sunscreen active ingredient in an amount effective to provide the composition with an SPF value of at least 4; and
(iii) water in an amount effective to provide the composition with a texture suitable for application to skin.

25. A method according to claim 24, wherein the step of applying comprises spray application of the sunscreen composition to skin.

26. A method according to claim 24, wherein the step of applying comprises hand application of the sunscreen composition to skin.
